(19) United States
(12) Patent Application Publication
(10) Pub. No.: US 2016/0256453 A1
(43) Pub. Date: Sep. 8, 2016

(54) OPIOID RECEPTOR ANTAGONIST FOR USE IN TREATING PATIENTS WITH SEVERE CONSTIPATION INDUCED BY HIGH OPIATE DOSAGE REGIMEN

(71) Applicant: Develco Pharma Schweiz AG, Pratteln (CH)

(72) Inventors: Helene Rey, Rosenau (FR); Olaf Mundszinger, Efringen-Kirchen (DE); Isabelle Golfier, Bantzenheim (FR); Silvia Jakob, Bernau (DE); Oliver Rusch, Basel (CH)

(21) Appl. No.: 14/828,043

(22) Filed: Aug. 17, 2015

(30) Foreign Application Priority Data
Mar. 6, 2015 (EP) 15170566.2

(51) Int. Cl. A61K 31/485

(52) U.S. Cl. CPC A61K 31/485

(57) ABSTRACT

The present invention relates to a pharmaceutical composition comprising an opioid receptor antagonist, or a derivative or a pharmaceutically acceptable salt thereof, as an active substance, wherein the composition releases the active substance in a prolonged manner. The composition that is suitable for an administration period of at least twelve-hours for the treatment of severe opioid-induced constipation in patients receiving a daily dosage of opioid equivalent to at least 80 mg of morphine.

### BACKGROUND

The present invention is in the field of medicine, particularly in the field of opioid therapy. The invention relates to a composition comprising an opioid receptor antagonist for use in treating a patient with severe opioid-induced constipation wherein the dosage of said opioid is equivalent to at least 80 mg morphine per day.

Opioids are the most effective analgesics widely used in patients with severe chronic pain. Their clinical efficacy is often burdened by adverse drug reactions that may influence drug adherence associated with early discontinuation, under-dosing, inadequate analgesia and reduced quality of life. One major health concern is constipation occurring in 80-90% of the patients on long-term opioid treatment. To avoid efficacy problems and specific signs and symptoms of the bowel dysfunction as haemorrhoid formation, rectal pain, burning or bowel obstruction, non-specific laxatives are frequently prescribed as stool softeners, bowel stimulant, or osmotic agents. Their clinical efficacy, however, is not undisputed and they may produce new problems. A more specific approach to avoid opioid induced constipation in patients with chronic pain is combination therapy with opioid antagonists that may prevent the undesired μ-opioid receptor-activation along the large intestine without compromising the centrally mediated analgesia or precipitating withdrawal effects. Suitable compounds are methylnaltrexone and naloxone that are either not absorbed from the gut lumen, do not pass the blood-brain barrier or are being inactivated along the “first-pass” absorption route. Subcutaneous methylnaltrexone (MNTX-SC) is approved in palliative care for the acute treatment of opioid induced constipation. The quaternary ammonium compound does not cross the blood-brain-barrier; consequently it does not antagonize the central effects and acts only in the periphery. After subcutaneous administration, it induces predominantly a short term laxative effect on constipation rather than the desired prevention of opioid induced bowel dysfunction.

There is also evidence that, despite its very low bioavailability, oral methylnaltrexone antagonizes the morphine-induced delay of intestinal transit time. In these studies, the pharmacodynamic effect and the systemic exposure were not correlated. Despite higher serum concentrations, methylnaltrexone as immediate-release formulation (MNTX-IR) provided lower efficacy compared to the extended-release formulation (MNTX-ER). However, the pharmacodynamics results with oral methylnaltrexone formulations are preliminary and may be misleading as the changes in intestinal motility were evaluated using the lactulose hydrogen breath test which is a measure for small intestinal transit (oro-cecal transit time, OCT). It has to be considered that 10 g of the osmotic active lactulose may exert own laxative effects and may accelerate OCT.

Opioid-induced constipation can be uncomfortable and very painful, and often leads to the discontinuation of the opioid-based therapy, and thus endangers the success of the treatment with the opioids especially in high doses. Since it can be assumed that the opioid-induced constipation is caused directly and locally over the entire intestine through binding to the opioid receptors, this side effect should be eliminated through the use of opioid antagonists. However, the use of opioid antagonists only makes sense if the antagonistic effect is limited to the intestine and does not cancel the main analgesic effect.

Naloxone is a suitable opioid antagonist for the treatment of opioid-induced constipation. Naloxone is rapidly and completely absorbed after oral administration and because the substance is subject to extensive first-pass metabolism, only small amounts of unmetabolised naloxone are available to the system.

The vast majority of the applied substance is found in blood in the form of inactive or only mildly active metabolites such as naloxone-3-glucuronide or beta-6-naloxol.

In suitable doses, naloxone is an ideal candidate for remedying opioid-induced constipation: in the intestine it is present as an active substance and can thus counter the paralysing effect of the opioid on the gastrointestinal tract, while after absorption it is largely metabolised during the first passage in the liver, and thereby becomes inactive. The analgesic effect of the opioids is thus not affected.

Since the paralysis does not only affect the duodenum and the upper part of the small intestine, but the entire gastrointestinal tract, the opioid-induced constipation cannot be treated successfully with a composition that releases the opioid antagonist rapidly. WO 2011/117306 discloses a two-layer tablet, which in one layer contains an opioid agonist, and in another layer an opioid antagonist, wherein the tablet quickly releases both active substances. The advantage of this double-layer is to suppress the side effects of the opioid agonist, but it does not focus on suppression of the opioid-induced constipation.

The combined preparation Targin® is available on the market and comprises a mixture of the opioid agonist oxycodone in the form of a hydrochloric salt, and the opioid antagonist naloxone also in the form of a hydrochloric salt. In this preparation, the active substances are released in a prolonged manner. It is therefore suitable for the parallel treatment of pain and opioid-induced constipation. However, this monolithic formulation has the disadvantage that the release rates of the two active substances are fixed. Individualised treatments are therefore difficult to optimise.

In addition, infusion solutions available on the market for the treatment of opioid poisoning are only naloxone combined preparations, in which naloxone and the opiate are present in a fixed proportion to each other. However, for the treatment of opioid-induced constipation, it would be desirable to have single agent naloxone preparations, since this would allow administering naloxone both independently of the nature of the opiate and in variable doses. The desired quantity of naloxone could therefore be applied, which would lead to an optimal treatment. Naloxone single agent preparations are described in the patent literature, such as in WO 98/25613 A2. However, the release of naloxone from these compositions is dependent on the ambient pH in the gastrointestinal tract. A uniform application of naloxone to the entire gastrointestinal tract, and therefore an optimal treatment, are thus not possible with such products.

Several confounding factors may aggravate constipation during palliative care (dehydration, confusion, drugs, immobility); opioids are a common cause. Prophylaxis can be considered when starting opioid therapy, although common constipation prophylaxis (e.g., fiber, fluids, exercise) may not be sufficient for patients receiving palliative care. In fact, fiber-based laxatives may be dangerous in those with fecal impaction and may result in impaction without adequate water intake.

Also, there is a need to address the problem of resistant opioid-induced constipation. Also, often first line treatments as for example with stool softeners and peristaltic stimulants fail.

And, the above products do not address the need of severely constipated patients. Such patients may for example be defined by their colorectal transit times (CTT). Such patients may have CTTs of 50 hours and more. This is a severe constipation.

Colon transit time is defined as the Whole gut transit (WGT) time minus the Oro-cecal transit time. The WGT time can be determined by any suitable method. However, in a preferred embodiment, the WGT time is determined using radio-opaque markers. Oro-cerecal transit time can be determined by any suitable method. Preferably, Oro-cecal transit time is determined by the sulfasalazine/sulfapyridine method (Gramatte, T. et al.; 1991, Int. J. Clin. Pharmacol. Ther. Toxicol; 29(4), 147-150).

### BRIEF DESCRIPTION OF THE DRAWINGS

FIG. 1 shows the release profile of naloxone from the composition according to the invention; example 1(•), example 2 (x).

### DESCRIPTION OF THE INVENTION

The present invention addresses those problems by a composition comprising an opioid receptor antagonist, or a pharmaceutically acceptable salt thereof, for use in treating a patient with severe constipation, wherein
(i) the antagonist in the composition is prepared in an oral extended release formulation;
(ii) the patient is characterized by a severe opioid-induced constipation;
(iii) wherein the patient is receiving an opiate treatment with a dosage equivalent to at least 80 mg of morphine per day.

The feature “the antagonist in the composition is prepared in an oral extended release formulation” means that the composition is an oral composition that releases the antagonist in a prolonged manner. A prolonged manner refers to the release of the antagonist over a 2, 4, 6, 8, 10, 12, 14, 16, 18, 20, 22, 24, 36, 48 hour period of time. In some instances it can be longer than 48 hours.

The phrase “receiving an opiate treatment” refers to a patient receiving at least one opiate treatment prior to or at the same time as the opioid receptor antagonist. In some instances, receiving an opiate treatment means more than one opiate treatment is received by the patient. For example, at least one opiate treatment can be prior to administration of the opioid receptor antagonist and at least one opiate treatment can be after administration of the opioid receptor antagonist.

The term “dosage equivalent” refers to a dosage of opiate that provides the same effects as a dosage of a second opiate. For example, naloxone can be administered in a dosage equivalent of at least 80 mg of morphine per day.

The phrase “severe opioid-induced constipation” refers to constipation caused by treatment with an opiate. In some instances, severe opioid-induced constipation is diagnosed by a doctor prior to a patient being administered an opioid receptor antagonist.

In preferred embodiments of the invention, the patient is receiving an opiate treatment with a dosage equivalent to at least 70, 80, 90, 100, 110, 120, 130, 140, 150, 160, 200, 240, 250, 300, 320, 400, or 500 mg of morphine per day. The higher the dosage of morphine equivalent, the more preferred is the embodiment.

In a more preferred embodiment of the invention the patient is receiving an opiate treatment with a dosage equivalent to 70 to 300 mg morphine, preferably equivalent to 75 to 270 mg morphine, more preferably 85 to 260 mg morphine and most preferably a dosage equivalent to 80 to 250 mg morphine per day.

In a preferred embodiment of the invention, the composition comprises an opioid receptor antagonist, or the pharmaceutically acceptable salt thereof, in a dose equivalent to 3 to 60 mg of naloxone hydrochloride, more preferably a dose equivalent to 8 to 48 mg of naloxone HCl, even more preferably a dose equivalent to 12 to 48 mg of naloxone HCl. In one embodiment the the compostion comprises methylnaltrexone bromide at a dosage of 150 mg, in an alternative embodiment the dosage of methylnaltrexone bromide is 500 mg.

An amount of an opioid or of an opioid antagonist is to be understood as the indicated amount±10%, ±8%, ±5%, ±3%, or more preferably ±1%.

In a preferred embodiment of the invention, the opioid receptor antagonist is a μ-receptor antagonist. In a more preferred embodiment, the opioid receptor antagonist is selected from the group of naloxone and its derivatives, in particular naloxone esters and sulfonates, further naltrexone, methylnaltrexone and pharmaceutically acceptable naltrexone derivatives or a pharmaceutically acceptable salt thereof. Most preferably, the opioid receptor antagonist is naloxone.

The aim of the present invention is to provides a solid oral pharmaceutical composition comprising an opioid receptor antagonist, or a pharmaceutically acceptable salt thereof, as an active substance, wherein the composition releases the active substance in a prolonged manner, and is suitable for an administration period of at least twelve-hours, for the treatment of severe opioid-induced constipation, wherein the dosage of said opioid is equivalent to at least 80 mg of morphine per day.

The in vitro release rate is determined using the paddle stirrer apparatus (apparatus 2) with the paddle stirrer method according to Ph. Eur. (European Pharmacopoeia, 7th edition, 3rd supplement, 2.9.3 “Dissolution test for solid dosage forms”, pages 3797-3803) at 75 rpm in 500 ml 0.1 N hydrochloric acid at 37° C. The amount of released active substance is preferably determined by UV-detection at 220 nm.

The opioid receptor antagonist is provided in an extended release formulation. Preferably the release rate of the opioid receptor antagonist is measured using the paddle stirrer method according to Ph. Eur. at 75 rpm in 500 ml 0.1 N hydrochloric acid at 37° C. In a preferred embodiment the release rate is 0% to 75% in 2 h, 3% to 95% in 4 h, 20% to 100% in 10 h, 30% to 100% in 16 h, 50% to 100% in 24 h, and of more than 80% in 36 h.

In a preferred embodiment the pharmaceutical composition comprises an opioid receptor antagonist such as naloxone, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 75% in 2 h, of 3% to 95% in 4 h, of 20% to 100% in 10 h, of 30% to 100% in 16 h, of 50% to 100% in 24 h, and of more than 80% in 36 h.

It was observed that the composition according to the invention, with its release profile, was suitable for an administration period of at least twelve-hours for the treatment of opioid-induced constipation. Accordingly it possesses a relatively high level of patient compliance.

The opioid-induced constipation which can be treated by the composition according to the invention can be caused by any opioid analgesic or opioid analgesic analogue, or by any of their salts or mixtures. Examples of such analgesics are the following: alfentanil, allylprodine, alphaprodine, anileridine, benzylmorphine, bezitramide, buprenorphine, butorphanol, clonitazene, codeine, besomorphine, dextromoramide, dezocine, diampromide, diamorphone, dihydrocodeine, dihydromorphine, dimenoxadol, Dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphane, lofentanil, meperidine, meptazinol, metazocine, methadone, metopone, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphane, phenazocine, phenoperidine, piminodine, piritramide, propheptazine, promedol, properidine, propoxyphene, sufentanil, tilidine, and tramadol, wherein hydrocodone, morphine, hydromorphone, oxycodone, buprenorphine, codeine, fentanyl, levorphanol, meperidine, methadone, levomethadone, and dextromethadone are particularly preferred according to the invention.

In a particularly preferred embodiment of the invention, the composition releases the active substance independently of the ambient pH of the gastrointestinal tract. This ensures that the entire gastrointestinal tract can be evenly and continuously supplied with the opioid receptor antagonist, or an acceptable salt thereof. A further optimisation of the treatment is thereby achieved. The pH-independent release of the active substance from the composition of the invention can be achieved through the choice of suitable pharmaceutical excipients that will be known to the person skilled in the art. Local pH values in the gastrointestinal tract are from about 1.2 (in the stomach), to about 6.8 in the colon.

The release of the active substance from the composition of the invention that is independent from the pH of the gastrointestinal tract is preferably understood to mean that the similarity factor f2 between a first in vitro release at a pH of 1.2 to 6.8 and a second in vitro release at any other pH of 1.2 to 6.8 is larger or equal to 50.

The similarity factor f2 is determined according to SHAH V. P., TSONG Y., SATHE P., & LIU J. P. (1998), “In vitro dissolution profile comparison-statistics and analysis of the similarity factor, f2”, Pharmaceutical Research, 15, 889-896. Specifically, the similarity factor f2 is calculated by the following formula:

$${f}_{2} = 50 * {log}_{10} ( {[ 1 + \frac{1}{n} {\sum }_{t = 1}^{n} {( {R}_{t} - {T}_{t} )}^{2} ]}^{0.5} * 100 )$$

In this equation, Rt and Tt represent the released quantities of active substance at time point t at the first and second pH. n is the number of time points. The f2 factor is determined under the following conditions: a) the minimal number of time points for one release is 3 (time point 0 is excluded); b) the time points for the first and the second pH should be equal; c) for each time point, and for each pH, the released quantity is indicated as the mean value of 12 measurements; d) no more than one mean value measured above a release of 85% can be taken into account for the calculation; e) the relative standard deviation or coefficient of variation of the release at a given pH should be smaller than 20% for the first time point and smaller than 10% for the second, and every subsequent time point.

In a further preferred embodiment of the invention, the composition comprises a matrix, which releases the active ingredient in a prolonged manner. The active substance can be released in a prolonged manner inexpensively, particularly when it is contained in a matrix that prolongs its release.

The composition according to the invention may comprise a matrix, which releases an opioid receptor antagonist such as naloxone, or a pharmaceutically acceptable salt thereof, in a prolonged manner. The matrix according to the invention is preferably a so-called scaffold matrix, which can be swelling or non-swelling, or can be a so-called eroding matrix. The matrix can also have properties of both scaffold and eroding matrixes.

In the case of a scaffold matrix, the active substance is incorporated into the matrix structure. The active substance is gradually dissolved by the digestive juices from the loaded scaffold matrix during the transport through the gastrointestinal tract. At the end of the process, the matrix scaffold is excreted in more or less unchanged form, or in a swollen form. In contrast, with an eroding matrix, the matrix is degraded, or eroded, which leads to active substance particles being exposed at the surface, and dissolved. The release rate therefore depends on the matrix degradation or erosion rate.

For the purpose of forming a largely stable scaffold matrix with an appropriate active substance release rate, a further preferred embodiment of the invention is a composition with a matrix that comprises one or several water-insoluble matrix-forming agents. Another embodiment of the invention is a composition with a matrix that comprises one or several water-soluble matrix-forming agents.

According to a further preferred embodiment of the invention, the matrix of the composition is water-insoluble. In an alternative embodiment of the invention, the matrix of the composition is water-soluble.

In another preferred embodiment of the invention, the matrix of the composition comprises one or several matrix-forming agents selected from the group consisting of cellulose esters, polyethylene oxide, polyvinylpyrrolidone/polyvinyl acetate mixtures, methacrylate-acrylate copolymers, waxes, fats such as glycerol esters, and fatty alcohols. The substance classes mentioned here are particularly suitable as matrix-forming agents for the composition of the invention. However, particularly preferred is the use of a mixture of polyvinyl acetate and polyvinylpyrrolidone, and/or a glycerol dibehenic acid ester as matrix-forming agent.

In a further preferred embodiment of the invention, the composition is free of film-coated, opioid receptor antagonist-containing particles, wherein the coating causes the prolonged release of the opioid receptor antagonist.

According to a further preferred embodiment of the invention, the composition can be formed by direct compression, since this is particularly inexpensive.

According to another preferred embodiment of the invention, the composition is in the form of a tablet, capsule, granule, a micro tablet, extruded particles or granules compressed into a tablet.

In a further preferred embodiment of the invention, the composition is designed as a once-a-day formulation, or a twice-a-day formulation.

Regarding the composition which is particularly suited for a twice-a-day administration, the present invention further relates to a solid oral pharmaceutical composition comprising an opioid receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 5% to 50% in 1 h, of 10% to 75% in 2 h, of 20% to 95% in 4 h, of 40% to 100% in 8 h, of more than 50% in 12 h, of more than 70% in 18 h, and of more than 80% in 24 h.

Regarding the composition which is particularly suited for a once-a-day administration, the present invention further relates to a solid oral pharmaceutical composition comprising an opioid receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 50% in 1 h, of 0% to 75% in 2 h, of 10% to 95% in 4 h, of 35% to 100% in 8 h, of 55% to 100% in 12 h, of 70% to 100% in 16 h and of more than 90% in 24 h.

Regarding the composition which is particularly suited for a once-a-day administration, the present invention further relates to a solid oral pharmaceutical composition comprising an opioid receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 0% to 30% in 1 h, of 0% to 40% in 2 h, of 3% to 55% in 4 h, of 10% to 60% in 8 h, of 20% to 75% in 12 h, of 30% to 88% in 16 h, of 50% to 100% in 24 h, and of more than 80% in 36 h.

Regarding the composition which is particularly suited for a once-a-day administration, the present invention further relates to a solid oral pharmaceutical composition comprising an opioid receptor antagonist, or a pharmaceutically acceptable salt thereof, as an active substance, wherein the composition releases the active substance in a prolonged manner, and the in vitro release rate of the active substance, measured using the paddle stirrer method according to Ph. Eur. at 75 rpm in 500 ml 0.1 N hydrochloric acid at 37° C., is of 10% to 30% in 1 h, of 17% to 37% in 2 h, of 27% to 47% in 4 h, of 40% to 60% in 8 h, of 50% to 70% in 12 h, of 60% to 80% in 16 h, and of 80% to 100% in 24 h.

In accordance with good patient compliance, a further preferred embodiment of the invention is a composition, wherein the composition is preferably a tablet or a capsule, which has an in vitro release rate of the active substance, measured using the paddle stirrer method according to Ph. Eur. at 75 rpm in 500 ml 0.1 N hydrochloric acid at 37° C., of 0% to 75% in 2 h, of 3% to 95% in 4 h, of 20% to 100% in 10 h, of 30% to 100% in 16 h, of 50% to 100% in 24 h, and of more than 80% in 36 h.

A further preferred embodiment of the invention is providing a composition that is suitable for the treatment of opioid-induced constipation for at least 12 h, provided that the composition has an in vitro release rate of the active substance of 0% to 50% in 2 h, of 5% to 95% in 4 h, of 20% to 90% in 10 h, of more than 70% in 18 h, and of more than 80% in 24 h.

The release rate is, in accordance with the invention, controlled by adjusting the mass ration of opioid receptor antagonist to matrix-forming agent. In a preferred embodiment, the mass ratio of opioid receptor antagonist to matrix-forming agent is 1:1, more preferably 1:2, more preferably 1:5, more preferably 1:10, more preferably 1:20, even more preferably 1:50, yet more preferably 1:75 and most preferably 1:100.

The composition of the invention is characterised in that through the prolonged release the concentration of the opioid receptor antagonist in the plasma is low. Its maximum plasma concentration (Cₘₐₓ) is about 20× lower during the active course compared to a composition without prolonged release, and about 100× lower compared with an intravenously administered composition.

The inhibition of the receptors over the active course is advantageous. In addition to providing the constipation prevention effect of the opioid receptor antagonist, the low bioavailability in the system also ensures a reduced likelihood and/or severity of the side effects.

Since the naloxone inhibitory concentrations (IC₅₀) for opioid receptors (μ, β and κ) are known, the assessment of the risk factor of a tablet can be calculated with the ratio IC₅₀/Cₘₐₓ. With the IC₅₀ of μ receptor, the value of IC₅₀/Cₘₐₓ for a tablet according to the invention with 48 mg of naloxone is 54. In general, the higher the value of IC₅₀/Cₘₐₓ, the lower the risk factor of the tablet according to the invention. Hereafter all values relating to the IC₅₀ are for the μ receptor.

In a preferred embodiment, the composition has an IC₅₀/Cₘₐₓ value of at least 30. In a more preferred embodiment, the composition has an IC₅₀/Cₘₐₓ value of at least 35. In an even more preferred embodiment, the composition has an IC₅₀/Cₘₐₓ value of at least 40. In the most preferred embodiment, the composition has an IC₅₀/Cₘₐₓ value of at least 50.

In a further embodiment, the composition additionally comprises at least one stabilizer, which protects the active substance. In a preferred embodiment, the at least one stabilizer is selected from the list comprising sulphur dioxide, sodium sulphite, sodium bisulphite, ascorbic acid and its derivatives and tocopherol, as well as its water- and fat-soluble derivatives, such as, for example, tocopherol acetate, sulphites, bisulphites and hydrogen sulphites of alkali, alkaline earth metals or other metals, paraben, BHA, BHT, gallates, as well as lower fatty acids, fruit acids, phosphoric acids, sorbic and benzoic acids as well as their salts, esters, derivatives and isomeric compounds, ascorbyl palmitate, lecithins, mono- and polyhydroxylated benzene derivatives, ethylenediaminetetraacetic acid and salts thereof, citraconic acid, cysteine, L-cysteine, conidendrin, diethyl carbonate, methylenedioxyphenols, cephalin, R,R′-dithiopropionic acid, biphenyl and other phenyl derivatives.

In a further embodiment, the composition additionally comprises at least one stabilizer, which protects the matrix. In a preferred embodiment, the at least one stabilizer is selected from the list comprising butylated hydroxytoluol, sulphur dioxide, sodium sulphite, sodium bisulphite, ascorbic acid and its derivatives and tocopherol, as well as its water- and fat-soluble derivatives, such as, for example, tocopherol acetate, sulphites, bisulphites and hydrogen sulphites of alkali, alkaline earth metals and other metals, paraben, BHA, BHT, gallates as well as lower fatty acids, fruit acids, phosphoric acids, sorbic and benzoic acids and their salts, esters, derivatives and isomeric compounds, ascorbyl palmitate, lecithins, mono- and polyhydroxylated benzene derivatives, ethylenediaminetetraacetic acid and their salts, citraconic acid, cysteine, L-cysteine, conidendrin, diethyl carbonate, methylenedioxyphenole, cephalin, R,R′-dithiopropionic acid, biphenyl and other phenyl derivatives.

In a further embodiment, the composition comprises at least one additive, wherein the additive is an emetic or a pungent agent drug. In a preferred embodiment, the composition comprises an additive, wherein this additive is a pungent agent, selected from the group comprising Allii sativi bulb, Asari rhizome cum herba, Calami rhizoma, capsici fructus {capsicum} capsici fructus acer (cayenne pepper), Rhizoma Curcumae Longae, Curcumae xanthorrhizae rhizoma, Galangae rhizoma, Semen Myristicae, Piperis nigri fructus (pepper), Sinapis albae (Erucae) Semen, Sinapis nigrae semen, Zedoariae rhizoma and Zingiberis rhizoma, preferably from the group consisting of capsici fructus (capsicum), capsici fructus acer (cayenne pepper) and Piperis nigri fructus (pepper).

In a preferred embodiment, the composition comprises at least one additive, wherein this additive is an emetic. In a preferred embodiment, the emetic is based on one or several substances from radix ipecacuanha (ipecac). In a preferred embodiment, the emetic is based on the substance emetine, in an alternative embodiment, the emetic is apomorphine.

In a further embodiment, the composition comprises a dye. In a preferred embodiment, the dye is selected from a group comprising red iron oxide, black iron oxide and indigo carmine.

In a further embodiment, the composition additionally comprises at least one non-steroid antirheumatic or an antihistamine.

In an alternative embodiment, the composition additionally comprises at least one water-soluble lubricant. In a preferred embodiment, the composition comprises at least one water-soluble lubricant selected from the group comprising adipic acid, fumaric acid, sodium benzoate and macrogol.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day. In some instances, the opioid receptor antagonist can be a μ-receptor antagonist. For example, the opioid receptor antagonist can be, but is not limited to, naloxone, methylnaltrexone, naltrexone, or a pharmaceutically acceptable salt thereof.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the composition comprises the opioid receptor antagonist, or the derivative or the pharmaceutically acceptable salt thereof, in a dose equivalent to 20 to 28 mg of naloxone.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the release rate of the opioid receptor antagonist measured using the paddle stirrer method at 75 rpm in 500 ml 0.1 N hydrochloric acid at 37° C., is 0% to 75% in 2 h, 3% to 95% in 4 h, 20% to 100% in 10 h, 30% to 100% in 16 h, 50% to 100% in 24 h, and of more than 80% in 36 h.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the composition releases the opioid receptor antagonist independently of the ambient pH of the gastrointestinal tract.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the composition can be a once-a-day formulation. In some instances, the composition can be a twice-a-day formulation. For example, the extended release formulation can have a 12-hour or a 24-hour release rate.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the opioid receptor antagonist is not naloxone.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the severe opioid-induced constipation is not caused by oxycodone.

Disclosed are methods of a treating severe constipation comprising administering a composition comprising an opioid receptor antagonist to a patient having severe opioid-induced constipation, wherein the composition is prepared in an extended release formulation, wherein the patient is receiving an opiate treatment with a dosage equivalent to at least 80 mg of morphine per day, wherein the extended release formulation is an oral extended release formulation.

### EXAMPLES

### Oral Composition

The following examples are used in conjunction with the drawing to illustrate the invention. It shows:

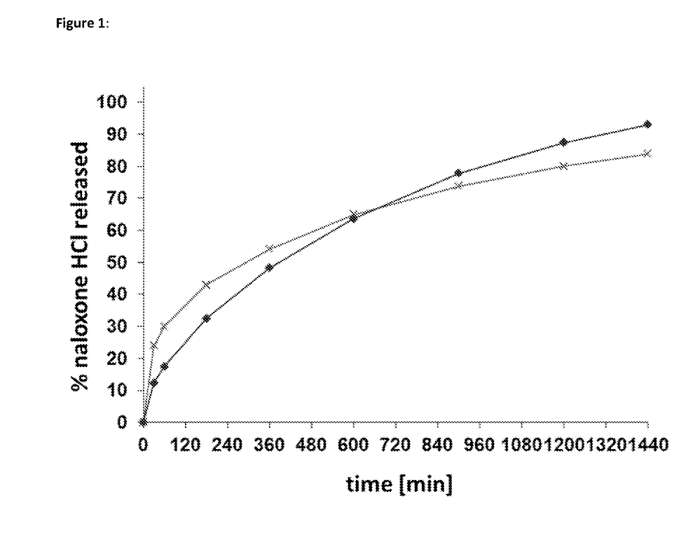

FIG. 1: Release profiles of the tablets according to examples 1 and 2.

### Example 1

Tablets with the following composition were produced:

| | | Weight |
|---|---|---|
| Substance | Function | [mg] |
| Naloxone hydrochloride | Active substance | 48.00 |
| Glycerol dibehenic acid ester | Release retardant | 204.64 |
| Colloidal silicon dioxide | Flow regulator | 19.00 |
| Magnesium stearate | Lubricant | 2.36 |
| | Total weight of the tablet | 274.00 |

The components naloxone hydrochloride and glycerol dibehenic acid ester were sieved and mixed together. First the sieved Colloidal silicon dioxide and then the magnesium stearate were mixed into the resulting mixture. The thus obtained mixture was pressed into a tablet using a conventional tablet pressing tool.

### Example 2

Tablets with the following composition were produced with the same method as in example 1:

| | | Weight |
|---|---|---|
| Substance | Function | [mg] |
| Naloxone hydrochloride | Active substance | 12.00 |
| Kollidon ® SR | Release retardant | 63.16 |
| Vivapur 200 | Filler | 7.00 |
| Colloidal silicon dioxide | Flow regulator | 1.24 |
| Magnesium stearate | Lubricant | 0.60 |
| | Total weight of the | 84.00 |
| | naloxone layer | |

Kollidon® SR consisting of 80 wt.-% polyvinyl acetate, 19 wt.-% povidone, 0.8 wt.-% sodium lauryl sulfate and 0.2 wt.-% Colloidal silicon dioxide.

### Release Profile

The in vitro release profiles of the tablets according to examples 1 and 2 were determined using a the paddle stirrer apparatus (apparatus 2) with the paddle stirrer method according to Ph. Eur. (European Pharmacopoeia, 7th edition, 3rd supplement, 2.9.3 “Dissolution test for solid dosage forms”, pages 3797-3803) at 75 rpm in 500 ml 0.1 N hydrochloric acid at 37° C. The amount of released naloxone was determined by UV-detection at 220 nm.

The in vitro release profiles of the tablets according to examples 1 (♦) and 2 (x) are shown in FIG. 1.

### Example 3

Coated two-layer tablets with the following composition were produced:

| | | Weight |
|---|---|---|
| Substance | Function | [mg] |
| Naloxone layer | | |
| Naloxone Hydrochloride | Active substance | 3.00 |
| Kollidon ® SR | Release retardant | 17.00 |
| Glycerol dibehenic acid ester | Release retardant | 4.75 |
| Colloidal silicon dioxide | Flow regulator | 0.60 |
| Magnesium stearate | Lubricant | 0.15 |
| | Total weight of the | 25.5 |
| | naloxone layer | |
| Placebo layer | | |
| Sugar pellets (diameter: 500-600 μm) | Carrier | 10.00 |
| Hypromellose | Filler | 10.00 |
| microcrystalline cellulose | Filler | 10.00 |
| Colloidal silicon dioxide | Flow regulator | 0.25 |
| Magnesium stearate | Lubricant | 0.25 |
| | Total weight of the | 30.50 |
| | placebo layer | |
| | Total weight of the | 56.00 |
| | two-layer tablet core | |
| Opadry ^{®} | Tablet coating | 3.00 |
| | Total weight of the | 59.00 |
| | two-layer tablet | |

The components of the naloxone layer, that is, naloxone hydrochloride, Kollidon® SR, glycerol dibehenic acid ester, colloidal silicon dioxide and magnesium stearate were sieved and blended together to form a first powdery mixture. Further, the components of the placebo layer: sugar pellets, hypromellose, microcrystalline cellulose, colloidal silicon dioxide and magnesium stearate were sieved and mixed together to form a second powdery mixture.

The first and the second mixture were pressed with a conventional two-layer tablet press to obtain the two-layer tablet core. The thus obtained two-layer tablet core was coated to obtain the two-layer tablet.

### Example 4

Coated two-layer tablets of the following composition were produced:

| | | Weight |
|---|---|---|
| Substance | Function | [mg] |
| Oxycodone layer | | |
| Sustained-release oxycodone pellets | Active substance | 80.00 |
| (containing 24 mg oxycodone HCl) | | |
| microcrystalline cellulose | Filler | 242.00 |
| Colloidal silicon dioxide | Flow regulator | 4.00 |
| Magnesium stearate | Lubricant | 4.00 |
| | Total weight of the | 330.00 |
| | oxycodone layer | |
| Naloxone layer | | |
| Naloxone hydrochloride | Active substance | 48.00 |
| microcrystalline cellulose | Filler | 84.00 |
| Kollidon ® SR | Release retardant | 204.00 |
| Colloidal silicon dioxide | Flow regulator | 10.00 |
| Magnesium stearate | Lubricant | 2.00 |
| | Total weight of the | 348.00 |
| | naloxone layer | |
| | Total weight of the | 678.00 |
| | two-layer tablet core | |
| Opadry ^{®} | Tablet coating | 22.00 |
| | Total weight of the | 700.00 |
| | two-layer tablet | |

The components of the oxycodone layer, that is, sustained release oxycodone pellets, microcrystalline cellulose, colloidal silicon dioxide and magnesium stearate were sieved and blended together to form a first powdery mixture.

Further, the components of the naloxone layer: naloxone hydrochloride, Kollidon® SR, colloidal silicon dioxide and magnesium stearate were sieved and mixed together to form a second powdery mixture.

The first and the second mixture were pressed with a conventional two-layer tablet press to obtain the two-layer tablet core. The thus obtained two-layer tablet core was coated with the coating material Opadry II® that had been dissolved in water at a temperature of 30° C. to 50° C. to obtain the two-layer tablet.

The sustained-release oxycodone pellets had the following composition and were prepared as known in the art:

| | | Weight |
|---|---|---|
| Substance | Function | [mg] |
| Oxycodone pellets | | |
| Oxycodone hydrochloride | Active substance | 3.00 |
| Pellets neutral | Carrier | 2.50 |
| Povidone | Binder | 0.50 |
| Retardant layer | | |
| Ethylcellulose | Retarding agent | 3.00 |
| Hydroxypropylcellulose | | 0.50 |
| Triethyl citrates | | 0.50 |
| | Total weight of the | 10.00 |
| | sustained release | |
| | oxycodone pellets | |

Kollidon® SR consists of 80 wt.-% polyvinyl acetate, 19 wt.-% povidone, 0.8 wt.-% sodium lauryl sulfate and 0.2 wt.-% Colloidal silicon dioxide.

Opadry II® consists of polyvinyl alcohol, iron oxide or titanium dioxide, Macrogol and talc.

### Example 5

Capsules containing micro tablets of 2 mm in diameter were produced as follow:

| | | Weight/capsule |
|---|---|---|
| Substance | Function | [mg] |
| Micro tablets | | |
| Methylnaltrexone bromide | Active substance | 150.00 |
| Povidone | | 16.50 |
| Colloidal silicon dioxide | Flow regulator | 2.25 |
| Magnesium stearate | Lubricant | 2.25 |
| Coating of the micro tablets | | |
| Ethylcellulose | Release retardant | 16.37 |
| Povidone | Binder | 4.91 |
| Propylene glycol | Plasticiser | 3.27 |
| Total weight of coated micro | | 195.55 |
| tablets/capsule | | |

The components methylnaltrexone bromide, povidone, colloidal silicon dioxide and magnesium stearate were mixed togethter and compressed on a rotary machine. Micro tablets were further coated with a solution of ethylcellulose, povidone and propylene glycol in a fluid bed processor in order to obtain a sustained release coating.

1. A method of a treating severe constipation comprising
administering a composition comprising an opioid receptor antagonist to a patient having severe opioid-induced constipation,
wherein the composition is prepared in a non-delayed, oral extended release formulation,
wherein the patient is receiving an opiate treatment with a dosage equivalent to at least 80 mg of morphine per day.

2. The method of claim 1, wherein the opioid receptor antagonist is a 11-receptor antagonist.

3. The method of claim 1, wherein the opioid receptor antagonist is naloxone, methylnaltrexone, naltrexone, or a pharmaceutically acceptable salt thereof.

4. The method of claim 1, wherein the composition comprises the opioid receptor antagonist, or the derivative or the pharmaceutically acceptable salt thereof, in a dose equivalent to 20 to 28 mg of naloxone.

5. (canceled)

6. The method of claim 1, wherein the composition releases the opioid receptor antagonist independently of the ambient pH of the gastrointestinal tract, wherein the composition comprises a hydrophobic polymer.

7. The method according to claim 1, wherein the composition is a once-a-day formulation.

8. The method according to claim 1, wherein the composition is a twice-a-day formulation.

9. The method of claim 1, wherein the opioid receptor antagonist is not naloxone.

10. The method of claim 1, wherein the severe opioid-induced constipation is not caused by oxycodone.

11. (canceled)
